SEQUENCE LISTING (4 sequences)

SEQ ID NO: 1 - DNA, 17 bp, Artificial Sequence
  Primer for amplification
ccccctggagctcgtgg

SEQ ID NO: 2 - DNA, 22 bp, Artificial Sequence
  Primer for amplification
gacagcagattctaaaacagtg

SEQ ID NO: 3 - DNA, 22 bp, Artificial Sequence
  Primer for amplification
aggatccgccatgacccccatc

SEQ ID NO: 4 - DNA, 45 bp, Artificial Sequence
  Primer for amplification
gcggccgctcaatgatgatgatgatgatgctcaccagccttggag

(19) United States
(12) Patent Application Publication
(10) Pub. No.: US 2009/0325187 A1
(43) Pub. Date: Dec. 31, 2009

(54) Immunomodulation

(76) Inventors: Torsten Witte, Hannover (DE); Torsten Matthias, Flonheim (DE)

Correspondence Address: GREER, BURNS & CRAIN, 300 S WACKER DR, 25TH FLOOR, CHICAGO, IL, 60606, US

(21) Appl. No.: 11/662,919

(22) PCT Filed: Sep. 15, 2004
(86) PCT No.: PCT/EP2004/052195

§ 371 Date: Jun. 4, 2009

(51) Int. Cl. G01N 33/53; C07K 14/00; C07K 16/18

(52) U.S. Cl. 435/7.1; 530/350; 530/387.9

(57) ABSTRACT

Medical use of ILT6 for modulating the immune response, as well as to pharmaceutical compositions containing ILT6.

In a further aspect, the present invention relates to the medical treatment of human beings using ILT6 to modulate their immune response, for example directed against heterologous and/or autologous antigens. Furthermore, the present invention relates to the analysis using ILT6 for diagnostic purposes and to diagnostic compositions that can be employed for that analysis.

The present invention relates to the modulation of an immune response in human beings. In greater detail, the present invention relates to the medical use of a compound for modulating the immune response, as well as to pharmaceutical compositions containing said compound.

In a further aspect, the present invention relates to the medical treatment of human beings using said compound to modulate their immune response, for example directed against heterologous and/or autologous antigen. Furthermore, the present invention relates to the analysis of said compound for diagnostic purposes and to diagnostic compositions that can be employed for that analysis or for the analysis of antibody cross-reactive with said compound.

### STATE OF THE ART

It is known to modulate an undesired activity of the immune system by generally suppressing the activity of the immune system. As an example, chemotherapy or the medical administration of hormones like cortisone are employed. Also known are genes encoded by the leucocyte Ig-like receptor cluster (LILR), containing at least thirteen genes encoding Ig-like transcripts (ILT) (Trowsdale et al., Immunol. Rev. 181, 20-38 (2001)).

ILTs are expressed by a variety of immune cells and it is known that ILT2 and ILT4 bind to HLA I molecules. It has been described by Chang et al., Nat. Immunol., 3: 237-243 (2002) that the expression of ILT3 and ILT4 may play a role in the generation of tolerance in T-cells. The regulation of the immune response towards antigen is exerted by ILTs by inhibiting or activating the cytolytic activity (Dietrich et al., J. Immunol., 166: 2514-2521 (2001)).

For ILT6, also termed LILRA3 or CD85e, it has been found that this is a soluble protein, i.e. it is not membrane-bound. Furthermore, Torkar et al., Eur. J. Immunol., 30: 3655-3662 (2000) have described that individuals lacking the seven 5′exons of the gene encoding ILT6 (deletion of a 6.7 kbp segment) exhibit a presence/absence variability for ILT6.

According to the DNA sequence, ILT6 has a certain homology to the ILT2 gene. The gene for ILT6 is localized on 19q13, a chromosomal region that has been shown to carry risk genes for multiple sclerosis (MS), a common demyelinating disease affecting the central nervous system. MS is characterized by a progressive destruction of the central nervous system by the individual's immune system, wherein the cellular immune response plays a significant role.

ILT2 is expressed on professional antigen presenting cells (APC) as well as on T-cells and natural killer cells. ILT2 binds to HLA I and acts as a receptor. ILT2 modulates the activity of T-cells and is participating in the induction of immunological tolerance by interacting with regulatory T-cells. For example, ILT2 participates in mediating tolerance of the immune system against mucosal antigens. It is furthermore presumed that ILT2 participates in the maternal/fetal immunotolerance during pregnancy.

### OBJECTS OF THE INVENTION

It is a first object of the present invention to provide a compound which can be used for medical purposes, e.g. in pharmaceutical compositions, to modulate the activity of the immune system of human beings. It is especially desired to provide a compound that predominantly exerts its effect on the cellular immune response. Furthermore, it is desired to provide a compound that can be used to activate or repress the activity of the immune system. In this respect, it is especially desired to provide a compound acting upon the activity of T-cells that form part of an acute immune response, preferably activated T-cells.

### GENERAL DESCRIPTION OF THE INVENTION

The present invention achieves the above-mentioned objects by providing ILT6, obtainable as the translation product from the human gene encoding ILT6. Alternatively, functional derivatives of the human ILT6 gene product are provided, obtainable for example by mutation, derivatization, deletion and/or fusion with itself or other peptides.

It has been found by the present inventors that ILT6, which is a soluble ILT, modulates the cellular immune response in a dose dependent manner. In detail, it has been found that low concentrations of ILT6, for example below 0.01 μg/mL, activate the proliferation of lymphocytes in an in vitro format, whereas concentrations higher than 0.01 μg/mL ILT6 inhibit the proliferation of lymphocytes in vitro.

ILT6 can be produced by known procaryotic and eucaryotic expression systems for heterologous protein expression from a DNA sequence derived from the human ILT6 gene. When expressing ILT6 in mammalian cells, the natural organization of the ILT6 gene including its intron sequences may be used, whereas for procaryotic expression systems or expression systems using yeasts or fungi, it is preferred to employ the DNA sequence devoid of intron sequences e.g. a DNA sequence derived from the cDNA of the processed transcript from the ILT6 gene.

In accordance with the above-mentioned objects of the invention, there is provided the human ILT6 gene product as well as its use for medical applications or as an active ingredient in pharmaceutical compositions suitable for indications of undesired immune responses like an autoimmune disease, e.g. MS or Sjögren's syndrome. Furthermore there is provided a diagnostic assay testing for the concentration of functionally active ILT6 in human beings, preferably in patients suffering from an autoimmune disease, e.g. MS or Sjögren's syndrome. In a further embodiment of the invention, there is provided a diagnostic assay testing for the concentration of antibody cross-reactive with ILT6, which antibody is present in patients, preferably in patients suffering from an autoimmune disease, e.g. MS or Sjögren's syndrome.

The present inventors have found that in a group of 751 patients with MS, homozygous ILT6 deficiency could be detected in 7.3%, but only in 3.8% of a control group, showing a statistically significant association with multiple sclerosis, especially the remitting relapsing MS, but not with primary progressive MS or secondary progressive MS. In controls, the distribution of homozygous and heterozygous ILT6 deletion did not differ from the Hardy-Weinberg equilibrium.

As many autoimmune diseases are polygenic, the deficiency of ILT6 in MS patients may be one of several contributing factors. The present inventors assume that the deficiency of ILT6 alone is not a prerequisite for the development of MS, as can be seen from its relation with remitting relapsing MS only. However, the differences of the association of ILT6 deficiency with RRMS (8.0%) to PPMS (7.0%) and SPMS (5.8%) are not statistically significant and may be due to the small sample sizes (RRMS, n=451; PPMS, n=129; SPMS, n=154). Accordingly, it cannot at present be concluded that ILT6 deficiency is characteristic for a specific subgroup of MS.

Although the mechanism for the action of ILT6 is not known, it is assumed that ILT6 may be acting as an agonist for a related ILT that binds to the same ligand, especially to MHC I. In the alternative, ILT6 could be acting as a soluble mediator between an antigen and cells of the immune system.

It could be shown that ILT6 predominantly exerts its immunomodulating effect on T-cells that have been activated. For example, monocytes and macrophages activated by interferon γ are immunomodulated by the presence of ILT6, however, without significantly influencing resting immune cells, e.g. resting monocytes, B-cells, T-cells or PBL (peripherial blood lymphocytes). For the purposes of immunomodulating the activity of activated immune cells, the natural translation product of the human ILT6 gene sequence can be used, for example expressed in animal cell culture from the structural gene for human ILT6. In the alternative, an identical translation product can be obtained from translation of a gene sequence in microorganisms which is derived from the human ILT6 gene sequence from which the intermittent intron sequences have deleted, optionally, signal sequences which are active in microorganisms used for the production of ILT6 protein may be added.

Furthermore, functional derivatives of ILT6 can be used for the purposes of this invention. Such functional derivatives comprise for example fusion proteins of different proteins or ILT6 itself with one or more regions of the natural ILT6 protein, and exchanges, deletions or substitutions of single amino acids or amino acid regions within the ILT6 protein. Examples for fusions with other peptides are tags useful for purification like the poly-histidine tag and antigenic sites which are detectable by known antibodies. Cloning and expression of functional derivatives of human ILT6 protein can be performed according to standard practice by a person skilled in the art without undue experimentation. The functional activity of ILT6 and its derivatives can be determined by a person skilled in the art by testing the immunomodulating characteristics of the protein, e.g. in a mixed lymphocyte assay. An example for such a functional test is given in example 3.

It is a specific advantage of ILT6 that it immunomodulates an active immune response i.e. an acute immune response by activated immune cells. Therefore, ILT6 can be employed for medical use, for example as an active component in pharmaceutical preparations for modulating an active immune response, for example for influencing the course of an acute phase of an immune response or a permanent immune response. In its preferred embodiment, the present invention employs ILT6 for medical use essentially without influencing resting immune cells. As an example, ILT6 can be used for treating immune responses due to for example the graft versus host disease or the host versus graft disease in transplant patients, or autoimmune diseases. Examples for autoimmune diseases are multiple sclerosis and Sjögren's syndrome, wherein ILT6 can be used to suppress the immune reaction, preferably during acute phases of the disease.

The administration of ILT6 for medical purposes can be obtained by injection, whether i.m., s.c., or i.v., or orally, using suitable formulations.

It has been found that the presence and concentration of ILT6 in body fluid, e.g. blood samples obtained from a human are in relation to the state of the human's immune system, e.g. the presence of an acute immune response.

Accordingly, the present invention in a further aspect provides a diagnostic assay to determine the concentration and/or activity of ILT6 in a sample of body fluid. As a result of the determination of the concentration and/or activity of ILT6, the state of the immune system of a human being can be determined, for example the presence or absence of an acute immune response. When relating the presence or absence of an acute immune response as determined by one of the analytical methods, to further symptoms or indications of the individual, the analytical methods can be used to determine the state or activity of an autoimmune disease, for example MS or Sjögren's syndrome.

In one embodiment, a diagnostic test relating to ILT6 is the analysis of the concentration of ILT6 in a sample of body fluid, e.g. blood, by an antibody based assay. One example for an antibody based assay is an ELISA format using at least one monoclonal or polyclonal antibody specific for ILT6 and a suitable detection system. The ELISA format may be non-competitive or competitive, for example using ILT6 protein expressed in eucaryotic or procaryotic cells as the competitor for ILT6 present in the sample of body fluid. Another format is a dot blot or Western blot of a sample of body fluid using the antibody directed against ILT6 for detecting ILT6 or cross-reactive derivatives thereof.

Antibody specific for ILT6 can be raised by immunization of a variety of laboratory animals, for example mice, rats and rabbits to obtain serum containing polyclonal antibody. Monoclonal antibody is obtainable by the hybridoma technique, using the fusion of spleen cells isolated from immunized laboratory animals with myeloma cells. A detailed description of production methods for polyclonal and monoclonal antibody is available from “Methods in Enzymology” and “Molecular Cloning, A Laboratory Manual by Sambrook, Maniatis, Fritsch, Cold Spring Harbour”.

In consideration of possible immune reactions directed against ILT6 in a human being suffering from an autoimmune disease or another undesired immune response and in view of possible immune reactions against ILT6 administered to a patient during a medical treatment, diagnostic methods and diagnostic compositions for the determination of such immune reactions against ILT6 are within the scope of the present invention.

Accordingly, in a further aspect the present invention provides for a diagnostic test for auto-antibodies directed against ILT6 and for antibodies directed against ILT6 administered to patients who do not express ILT6. In this respect, ILT6 or functional derivatives thereof are provided for use in an immunological assay to determine the presence and concentration of antibody in a sample of body fluid cross-reactive with ILT6. As described above, ILT6 and its functional derivatives may be produced by heterologous gene expression or, alternatively, synthetically by peptide synthesis, the derivative preferably comprising the epitopes cross-reactive with auto-antibody or antibody directed against ILT6 only or repeatedly.

The diagnostic test may be in the format of an ELISA, blot or any other immunological assay using ILT6 or an immunological equivalent thereof as the capture protein to specifically adsorb antibody cross-reactive with ILT6.

### DETAILED DESCRIPTION OF THE INVENTION

The invention is now described in greater detail with relation to the accompanying figures, wherein

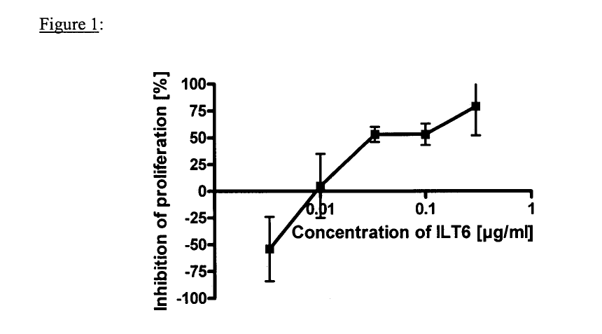

FIG. 1 is a graph showing the inhibition and stimulation, respectively, of peripheral blood mononuclear cells depending on the concentration of ILT6.

For Sjögren's syndrome, ILT6 deficiency was present in 9% of patients, which is a statistically significant association with the disorder. In contrast, the ILT6 deficiency is not associated with SLE and scleroderma.

### EXAMPLE 1

### Cloning of the Human ILT6 Gene

Genomic DNA was extracted from peripheral whole blood using the QiaAmp DNA Minikit (Qiagen, Hilden, Germany). Using PCR primers 5′CCC CCT GGA GCT CGT GG 3′ (Seq. ID No. 1) and 5′GAC AGC AGA TTC TAA AAC AGT G 3′ (Seq. ID No. 2), the complete ILT6 gene comprising 1150 base pairs was amplified in a in the PCR reaction using 10 pmoles of each primer, 50 ng genomic DNA in 1×PCR buffer containing 1.5 mM MgCl₂, 200 μM dNTPs, 2.5 units Taq polymerase according to the manufacturer's instructions.

Thermocycling used 95° C. for 15 minutes, 30 cycles of 94° C. for 45 seconds, 64° C. for 55 seconds and 72° C. for 55 seconds, and a final extension at 72° C. for 10 minutes. Products were separated by electrophoresis (1.5% agarose), staining which ethidium bromide and detection under UV.

For sequencing the PCR products were cloned into the PCR 2.1 vector using the TA cloning kit (Invitrogen, Karlsruhe).

Statistical analysis was performed with Fisher's exact test, regarding only associations below 0.05 as significant.

### EXAMPLE 2

### Production of ILT6

For production of ILT6 protein, the cDNA encoding human ILT6 was synthesized using RT-PCR on complete RNA isolated from human peripheral whole blood according to known methods, for example using the QIAamp RNA blood mini kit (Qiagen, Hilden, Germany) according to the manufacturer's instructions.

For expression and later purification, the cDNA encoding the complete exons for human ILT6 without interfering intron sequences additionally received a fused sequence encoding a poly-histidine tag in 3′. This is one example for a functional derivative of human ILT6, retaining its biological activity.

For RT-PCR, the following primers were used on cellular RNA: 5′ AGG ATC CGC CAT GAC CCC CAT C 3′ (Seq. ID No. 3) containing an additional BamH1 site 5′ to the start codon and 5′GCG GCC GCT CAA TGA TGA TGA TGA TGA TGC TCA CCA GCC TTG GAG 3′ (Seq. ID No. 4), encoding a histidine tag 5′ to the stop codon and containing a NotI site 3′ to the stop codon. The resultant gene product is subsequently termed ILT6::poly-His.

Expression of the fusion peptide ILT6::poly-His was cloned into expression vector pBacPak8 (Clontech) using restriction sites BanH1 and Not1 subsequent to ligating the PCR product into vector pCR2.1 (Invitrogen) and sequencing. After transfer into bacculovirus, cultivated SF9 cells were infected. For expression, cells were grown in TNM-FH medium (described in Hink et al., Nature 226: 466-467 (1970) as obtainable from 900 mL Graces medium, combined with 80 mL fetal bovine serum, 3.0 g lactalbumin hydrolysate, 3.0 g Yeastolate, stirred vigorously until powdered ingredients dissolve; if necessary, adjust pH to 6.40-6.45 by addition of 1.0 N KOH (increase) or 1.0 N HCl (decrease); if necessary, adjust osmotic pressure to 360-380 milliosmols with anhydrous D-glucose (increase) or water (decrease) (obtained from Sigma-Aldrich, product No. T3285) and induced by infection for a period of 5-6 days at 37° C. in a 5% CO₂ atmosphere until plaque formation.

After harvesting cells, the protein was purified from the cellular lysate making use of the His-tag in nickel chelate chromatography. The eluted ILT6::poly-His was eluted from the nickel chelate column and used for immunomodulating experiments.

### EXAMPLE 3

### In Vitro Activity of ILT6 on the Immune System

Recombinantly produced ILT6::poly-His obtained according to Example 3 was used for modulating the activity of peripheral blood lymphocytes (PBL) in a mixed lymphocyte reaction. The mixed lymphocyte reaction comprised 100,000 PBL/well obtained from a healthy first donor that were stimulated by the addition of 20,000 PBL obtained from a second healthy donor which had been irradiated before their addition.

Recombinant ILT6 obtained according to Example 3 was added to final concentrations between 0.33 μg/mL and 0.0033 μg/mL.

For monitoring the proliferation of the PBL obtained from the first donor, ³H-thymidine was added after 72 hours and the test reactions were harvested after a total of 96 hours. For comparison, protein Ro expressed and purified by the method according to Example 3, replacing the ILT6 gene sequence by the Ro gene sequence, was used.

In FIG. 1, the inhibition of proliferation of peripheral blood mononuclear cells in a mixed lymphocyte reaction by addition of ILT6 is shown. The average and standard deviations obtained from experiments with three different donors of stimulating PBL is plotted.

The results demonstrate that high concentrations of ILT6, especially concentrations equal to or above 0.033 μg/mL blocked proliferation of activated PBL, whereas low concentrations of ILT6, especially below 0.0033 μg/mL, were stimulating their proliferation. When using Ro protein in comparative assays, essentially no inhibition of proliferation nor their activation was found.

When examining the morphology of PBL microscopically, it was detected that concentrations of ILT6 inhibiting the proliferation of PBL did not lead to toxic effects to PBL but rather to an arrest of their proliferation.

### EXAMPLE 5

### Direct Modulation of T-Cells by ILT6

For monitoring the intracellular calcium concentrations in T-cells treated with ILT6 at varying concentrations, the following assay was used: PBL or Jurkat T-cells were loaded with 20 μM of the visible light-excitable Ca²⁺ indicator fluoro-h fluorochrome (CAS 121714-22-5/Glycine, N-[4-[6-[(acetyloxy)methoxy]-2,7-dichloro-3-oxo-3H-xanthen-9-yl]-2-[2-[2-[bis[2-[(acetyloxy)methoxy]-2-oxyethyl]amino]-5-methylphenoxy]ethoxy]phenyl]-N-[2-[(acetyloxy)methoxy]-2-oxyethyl]-, (acetyloxy)methyl ester (obtained from Molecular Probes, c/o Invitrogen, Karlsruhe, Germany, under catalog No. F1241) for 30 min at 37° C., then washed gently three times in PRMI medium and once with PBS (phosphate buffered saline). Finally, calcium flux was induced by addition of ILT6 at various concentrations. As a positive control, 10 μM Ionomycin was added to a separate test sample. Intracellular release of calcium was measured at 400 nm in a FACScalibur (Becton Dickinson) fluorescence activated cell sorter.

It was found that presence of ILT6 induces the intracellular liberation of calcium. This demonstrates that ILT6 directly acts on T-cells.

In consideration of the observation that IFN-gamma is detectable at increased levels during acute phases of MS, it can be deduced from the direct activation of the production of ILT6 by IFN-gamma that ILT6 participates physiologically in the course of inflammatory diseases like autoimmune diseases, e.g. multiple sclerosis. However, the increased production of ILT6 during inflammatory states does not impair the medical application of ILT6 according to the invention.

1. A human ILT6 protein selected from the natural translation product of the human ILT6 gene and functional derivatives thereof, for medical use.

2. The ILT6 according to claim 1, wherein the medical use is directed against diseases selected from autoimmune diseases and undesired immune responses.

3. The ILT6 according to claim 2, wherein the autoimmune disease is multiple sclerosis, Sjögren's syndrome or an autoimmune disease having acute phases.

4. The ILT6 according to claim 2, wherein the undesired immune response is the graft versus host disease or the host versus graft disease in transplant patients.

5. The ILT6 according to claim 1, wherein the medical use is the reduction of an immune response.

6. The ILT6 according to claim 1, wherein the immune response is the cellular immune response.

7. The ILT6 according to claim 2, wherein the immune response is the humoral immune response.

8. The ILT6 according to claim 1, wherein ILT6 is the natural translation product from the human ILT6 gene sequence or a functional derivative thereof expressed in eucaryotic or procaryotic cells.

9. A pharmaceutical composition comprising ILT6 according to claim 1.

10. A method for analyzing an immune response or the state of activation of the immune system of a human, comprising determining the concentration of ILT6 in a sample of body fluid obtained from the human.

11. The method according to claim 10, wherein in that the determination of ILT6 is an antibody based assay.

12. The method according to claim 10, wherein the human suffers from an autoimmune disease.

13. The method according to claim 12, wherein the autoimmune disease is selected from the group comprising multiple sclerosis and Sjögren's syndrome and autoimmune diseases having acute phases.

14. An antibody specific for ILT6, characterized in being suitable in the method according to claim 10.

15. A method for determining antibody cross-reactivity with ILT6 in a sample of body fluid.

16. The ILT6 protein or a derivative thereof having immunological reactivity derivable from ILT6, for use in the method according to claim 15.

17. A method for analyzing an immune response or the state of activation of the immune system of a human, comprising determining the in vitro activity of ILT6 contained in a sample of body fluid obtained from the human in a mixed lymphocyte reaction.
